(19) United States
(12) Patent Application Publication
(10) Pub. No.: US 2025/0277784 A1
(43) Pub. Date: Sep. 4, 2025

(54) EXPRESSION SYSTEMS FOR THE ALPHA6BETA4 NICOTINIC ACETYLCHOLINE RECEPTOR AND METHODS OF USE THEREOF

(71) Applicant: Janssen Pharmaceutica NV, Beerse (BE)

(72) Inventors: David S Bredt, La Jolla, CA (US); Shenyan Gu, San Diego, CA (US); Jose Matta, San Diego, CA (US); Daniel Knowlan, San Diego, CA (US); Weston Davini, San Diego, CA (US); James Limberis, San Diego, CA (US)

(21) Appl. No.: 18/288,018

(22) PCT Filed: Apr. 22, 2022
(86) PCT No.: PCT/EP2022/060765

§ 371 Date: Oct. 23, 2023

Related U.S. Application Data
(60) Provisional application No. 63/178,835, filed on Apr. 23, 2021.

(51) Int. Cl. G01N 33/50; C07K 14/47; C07K 14/725; C12N 9/10; G01N 33/94

(52) U.S. Cl. CPC G01N 33/5041; C07K 14/4702; C07K 14/7051; C12N 9/1029; C12N 9/13; G01N 33/944; C12Y 203/01006; C12Y 208/02002; G01N 2333/4703; G01N 2333/705; G01N 2333/91057; G01N 2333/91194

(57) ABSTRACT

Disclosed herein are isolated recombinant cells for the expression of α6β4 nicotinic acetylcholine receptor (nAChR) and methods of use thereof.

### CROSS REFERENCE TO RELATED APPLICATION

This application claims the benefit of U.S. Provisional Application 63/178,835, filed on Apr. 23, 2021, which is incorporated by reference herein in its entirety.

### FIELD OF INVENTION

This invention relates to isolated recombinant cells for the expression of α6β4 nicotinic acetylcholine receptor (nAChR) and methods of use thereof.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Feb. 28, 2022, is named PRD4127WOPCT1_SL.txt and is 24,576 bytes in size.

### BACKGROUND OF INVENTION

Nicotine has analgesic properties owning to actions on nicotinic acetylcholine receptors (nAChRs) (Hone et al., Nicotinic Acetylcholine Receptors in Neuropathic and Inflammatory Pain, FEBS Lett. 2018 April; 592 (7):1045-1062. doi: 10.1002/1873-3468.12884). Epibatidine, is a general nAChR agonist and a powerful analgesic (Daly et al., Alkaloids from Frog Skin: the Discovery of Epibatidine and the Potential for Developing Novel Non-Opioid Analgesics Nat Prod Rep. 2000 April; 17(2):131-5. doi: 10.1039/a900728h). Furthermore, a pan-nAChR agonist ABT-594 showed robust efficacy for diabetic neuropathy in a phase 2 clinical trial. Unfortunately, ABT-594 had unacceptable side effects and its precise molecular target was unknown (Rowbotham et al., A randomized, double-blind, placebo-controlled trial evaluating the efficacy and safety of ABT-594 in patients with diabetic peripheral neuropathic pain, Pain. 2009 December; 146(3):245-252. doi: 10.1016/j.pain.2009.06.013. Epub 2009 Jul. 24). Subsequently, a genomics screen of mRNA expression in dorsal root ganglion tissue from outbred mouse strains identified that 6 nAChR subunit levels inversely correlate with pain responses in a spare nerve injury model (Wieskopf et al., The nicotinic α6 subunit gene determines variability in chronic pain sensitivity via cross-inhibition of P2X2/3 receptors, Sci Transl Med. 2015 May 13; 7(287):287ra72. doi: 10.1126/scitranslmed.3009986). Accordingly, analgesic effects of nicotine following both inflammatory and neuropathic injuries are absent in α6 knockout mice. Furthermore, human postoperative pain and temporomandibular disorder are affected by a polymorphism in the CHRNA6 (α6) promoter (Wieskopf et al., The nicotinic α6 subunit gene determines variability in chronic pain sensitivity via cross-inhibition of P2X2/3 receptors, Sci Transl Med. 2015 May 13; 7(287):287ra72. doi: 10.1126/scitranslmed.3009986). Taken together, these results provide compelling evidence that agonism on α6 in spinal neuron, which pair with β4 subunits can relieve neuropathic pain and is an appealing non-opioid target (Hone et al., Nicotinic Acetylcholine Receptors in Neuropathic and Inflammatory Pain, FEBS Lett. 2018 April; 592(7):1045-1062. doi: 10.1002/1873-3468.12884).

Drug discovery on human α6β4 receptors has however not been possible, as these receptors have been refractory to expression in recombinant cell lines (Yang et al., Mysterious α6-containing nAChRs: function, pharmacology, and pathophysiology, Acta Pharmacologica Sinica, 2009 June; 30(6): 740-751)

There still is a need to develop a cell line that expresses these receptors robustly and can be used for drug discovery.

### BRIEF SUMMARY OF THE INVENTION

Provided herein are isolated recombinant cells comprising: a) a heterologous nucleic acid encoding an α6 subunit of an α6β4 nicotinic acetylcholine receptor (nAChR); b) a heterologous nucleic acid encoding a β4 subunit of an α6β4 nAChR; and c) at least one heterologous nucleic acid selected from the group consisting of a heterologous nucleic acid encoding beta associated regulatory protein (BARP), a heterologous nucleic acid encoding choline O-acetyltransferase (CHAT), and a heterologous nucleic acid encoding sulfotransferase family 2B member 1 (SULT2B1).

In one embodiment of the isolated recombinant cells, the recombinant cell comprises the heterologous nucleic acid encoding BARP, the heterologous nucleic acid encoding CHAT, and the heterologous nucleic acid encoding SULT2B1.

In a further embodiment of the isolated recombinant cells, the recombinant cell is a mammalian cell.

In a yet further embodiment of the isolated recombinant cells, the mammalian cell is selected from the group consisting of a human embryonic kidney 293T (HEK293T) cell, a HEK293F cell, a HeLa cell, a Chinese hamster ovary (CHO) cell, a NIH 3T3 cell, a MCF-7 cell, a Hep G2 cell, a baby hamster kidney (BHK) cell, and a Cos7 cell.

In a yet further embodiment of the isolated recombinant cells, the α6 subunit of the α6β4 nAChR comprises an amino acid sequence with at least 95% identity to the amino acid sequence of SEQ ID NO: 1.

In a yet further embodiment of the isolated recombinant cells, the β4 subunit of the α6β4 nAChR comprises an amino acid sequence with at least 95% identity to the amino acid sequence of SEQ ID NO: 2.

In a yet further embodiment of the isolated recombinant cells, the BARP comprises an amino acid sequence with at least 95% identity to the amino acid sequence of SEQ ID NO: 3.

In a yet further embodiment of the isolated recombinant cells, the CHAT comprises an amino acid sequence with at least 95% identity to the amino acid sequence of SEQ ID NO: 4.

In a yet further embodiment of the isolated recombinant cells, the SULT2B1 comprises an amino acid sequence with at least 95% identity to the amino acid sequence of SEQ ID NO: 5.

Further provided herein are methods for identifying agonists, antagonists, or positive allosteric modulators of α6β4 nAChR, the method comprising: a) contacting the isolated recombinant cell described above with an agent; and b) determining the activity of the α6β4 nAChR of the isolated recombinant cell, wherein the agent is identified as an agonist or positive allosteric modulator (PAM) if the agent enhances the activity of the α6β4 nAChR and the agent is identified as an antagonist if the agent decreases the activity of the α6β4 nAChR as compared to the activity of the α6β4 nAChR when the isolated recombinant cell was not contacted with the agent.

In a further embodiment of the method, step b) comprises determining calcium flux of the isolated recombinant cell, wherein the agent is identified as an agonist if the agent enhances the calcium flux as compared to the calcium flux when the isolated recombinant cell was not contacted with the agent.

In a yet further embodiment of the method, step b) comprises determining calcium flux and nicotine-evoked calcium flux of the isolated recombinant cell, wherein the agent is identified as an PAM if the agent does not enhance calcium flux and enhances the nicotine-evoked calcium flux as compared to the calcium flux and nicotine-evoked calcium flux when the isolated recombinant cell was not contacted with the agent

In a yet further embodiment of the method, step b) comprises determining nicotine-evoked calcium flux of the isolated recombinant cell, wherein the agent is identified as an antagonist if the agent decreases the nicotine-evoked calcium flux as compared to the nicotine-evoked calcium flux when the isolated recombinant cell was not contacted with the agent.

In a yet further embodiment of the method, the isolated recombinant cell is incubated at about 25° C.-35° C. for about 20-50 hours prior to being contacted with the agent.

In a yet further embodiment of the method, the agent is a small molecule or peptide.

Yet further provided herein is a kit comprising (i) the isolated recombinant cell as described above and (ii) instructions for use.

### BRIEF DESCRIPTION OF THE DRAWINGS

FIG. 1 is a plot of FLIPR signal of the recombinant cells after incubation with agonists of α6β4 nAChR at various concentrations.

FIG. 2 is a plot of FLIPR signals of the recombinant cells after first incubation with methyllycaconitine (MLA) (a known antagonist of α6β4 nAChR) at various concentrations and second incubation with nicotine.

FIG. 3 is a graph showing functional expression of α6β4 nAChR when co-expressed with different chaperon proteins.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2 111.03.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1992)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Isolated Recombinant Cells and Methods of Making Recombinant Cells

The inventions disclosed herein are based, at least in part, on the unexpected finding that co-expressing certain chaperone proteins with α6β4 nicotinic acetylcholine receptor (nAChR) in cells generated cells that highly express α6δ4 nicotinic acetylcholine receptor (nAChR), making the cells useful for drug discovery. Provided herein are methods of making recombinant cells expressing α6β4 nicotinic acetylcholine receptor (nAChR) and isolated recombinant cells for the expression of α6β4 nicotinic acetylcholine receptor (nAChR).

As used herein, the terms “α6β4 nicotinic acetylcholine receptor”, “α6β4 nAChR”, “alpha6beta4 nicotinic acetylcholine receptor”, and “alpha6beta4 nAChR” are used interchangeably and refer to the α6β4 nicotinic acetylcholine receptor protein, preferably the human α6β4 nAChR, which is a member of a protein family of cholinergic receptors.

α6β4 nAChR is a ligand-gated ion channel composed of α6 and β4 subunits. The α6 subunit is encoded by the gene CHRNA6 (NM_004198) and the β4 subunit is encoded by the gene CHRNB4 (NM_000750). When expressed together, the subunits co-assemble to form a heteromeric nAChR (See e.g., Hone et al., Nicotinic acetylcholine receptors in dorsal root ganglion neurons include the α6β4* subtype, FASEB J. 2012; 26(2):917-926).

“Recombinant cells” refers to one or more individual cells as well as to a recombinant cell line in which the cells are heterologously expressing protein(s). As used herein, “heterologous expression” of a protein in a cell refers to modifying the cell to express the protein by introducing an exogenous nucleic acid into the cell, e.g., an exogenous nucleic acid that encodes the protein to be expressed. A “heterologous nucleic acid” refers to a nucleic acid exogenous to a cell that is introduced into the cell. In some embodiments, the heterologous nucleic acid is DNA. In some embodiments, the heterologous nucleic acid is RNA. Heterologous expression of a protein in a cell can be achieved using a variety of methods. For example, an expression vector comprising a nucleic acid encoding the protein that is operably linked to a nucleic acid encoding a promoter capable of driving expression of the protein (e.g., a constitutive promoter) may be introduced into the cell.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications.

In a general aspect, the invention relates to methods of making or generating cells expressing α6β4 nicotinic acetylcholine receptor (nAChR) that are useful for drug discovery. In one embodiment, the method comprises introducing into a cell a nucleic acid encoding an α6 subunit of an α6β4 nAChR, a nucleic acid encoding a β4 subunit of an α6β4 nAChR, and a nucleic acid encoding at least one protein selected from: beta associated regulatory protein (BARP), choline O-acetyltransferase (CHAT), and sulfotransferase family 2B member 1 (SULT2B1). The nucleic acid(s) encoding the α6 subunit of an α6β4 nAChR, β4 subunit of an α6β4 nAChR, BARP, CHAT, and/or SULT2B1 can be in an expression vector (e.g., in a single expression vector or in separate expression vectors). In some embodiments, the nucleic acid encoding the α6 subunit of an α6β4 nAChR, the β4 subunit of an α6β4 nAChR, BARP, CHAT, and/or SULT2B1 is operably linked to a promoter capable of driving expression of the respective protein. In some other embodiments, each of the nucleic acids encoding the α6 subunit of an α6β4 nAChR, β4 subunit of an α6β4 nAChR, BARP, CHAT, and SULT2B1 is operably linked to a promoter capable of driving expression of the respective protein. In some embodiments, the promoter is a constitutive promoter.

In another aspect, the method comprises introducing into a cell a nucleic acid encoding an α6 subunit of an α6β4 nAChR, a nucleic acid encoding a β4 subunit of an α6β4 nAChR, and a nucleic acid encoding at least one protein selected from: BARP, CHAT, and SULT2B1, wherein the cell generated by the method expresses α6β4 nAChR and/or the at least one protein at an increased level compared to the same cell without the nucleic acid encoding the α6 subunit of an α6β4 nAChR, the nucleic acid encoding the β4 subunit of an α6β4 nAChR, and the nucleic acid encoding the at least one protein introduced.

In another aspect, the invention relates to cells genetically modified to express at least one protein selected from: BARP, CHAT, and SULT2B1, wherein the genetically modified cell expresses the at least one protein at an increased level relative to the expression of the same protein in the unmodified cell under the same (or substantially the same) conditions. In one embodiment, the cells are genetically modified to express each of BARP, CHAT, and SULT2B1, wherein genetically modified cell expresses BARP, CHAT, and SULT2B1 at an increased level relative to the expression of BARP, CHAT, and SULT2B1, respectively, in the unmodified cell under the same (or substantially the same) conditions.

In another aspect, the invention relates to cells genetically modified to express an α6 subunit of an α6β4 nAChR, a β4 subunit of an α6β4 nAChR, and at least one protein selected from: BARP, CHAT, and SULT2B1, wherein the genetically modified cell expresses the at least one protein at an increased level relative to the expression of the same protein in the unmodified cell under the same (or substantially the same) conditions. In one embodiment, the cells are genetically modified to express an α6 subunit of an α6β4 nAChR, a β4 subunit of an α6β4 nAChR, and each of BARP, CHAT, and SULT2B1, wherein genetically modified cell expresses BARP, CHAT, and SULT2B1 at an increased level relative to the expression of BARP, CHAT, and SULT2B1, respectively, in the unmodified cell under the same (or substantially the same) conditions.

In another aspect, the invention relates to isolated recombinant cells comprising at least one expression vector selected from the group consisting of an expression vector comprising a nucleic acid sequence encoding BARP, an expression vector comprising a nucleic acid sequence encoding CHAT, and an expression vector comprising a nucleic acid sequence encoding SULT2B1. In one embodiment, the invention relates to isolated recombinant cells comprising an expression vector comprising a nucleic acid sequence encoding beta associated regulatory protein (BARP), an expression vector comprising a nucleic acid sequence encoding choline O-acetyltransferase (CHAT), and an expression vector comprising a nucleic acid sequence encoding sulfotransferase family 2B member 1 (SULT2B1).

In another aspect, the invention relates to isolated recombinant cells comprising an expression vector comprising a nucleic acid encoding an α6 subunit of an α6β4 nAChR, an expression vector comprising a nucleic acid encoding a β4 subunit of an α6β4 nAChR, and at least one expression vector selected from the group consisting of an expression vector comprising a nucleic acid encoding BARP, an expression vector comprising a nucleic acid encoding CHAT, and an expression vector comprising a nucleic acid encoding SULT2B1. In one embodiment, the invention relates to isolated recombinant cells comprising an expression vector comprising a nucleic acid encoding an α6 subunit of an α6β4 nAChR, an expression vector comprising a nucleic acid encoding a β4 subunit of an α6β4 nAChR, an expression vector comprising a nucleic acid encoding BARP, an expression vector comprising a nucleic acid encoding CHAT, and an expression vector comprising a nucleic acid encoding SULT2B1.

In any one of the embodiments described herein, the nucleic acid(s) encoding any one or more of the α6 subunit of an α6β4 nAChR, β4 subunit of an α6β4 nAChR, BARP, CHAT, and SULT2B1 can be in a single expression vector or in separate expression vectors.

In another aspect, the invention relates to isolated recombinant cells comprising: a) a heterologous nucleic acid encoding an α6 subunit of an α6β4 nAChR; b) a heterologous nucleic acid encoding a β4 subunit of an α6β4 nAChR; and c) at least one heterologous nucleic acid selected from the group consisting of a heterologous nucleic acid encoding BARP, a heterologous nucleic acid encoding CHAT, and a heterologous nucleic acid encoding SULT2B1.

In one embodiment, the invention relates to isolated recombinant cells comprising: a) a heterologous nucleic acid encoding an α6 subunit of an α6δ4 nAChR; b) a heterologous nucleic acid encoding a β4 subunit of an α6β4 nAChR; c) a heterologous nucleic acid encoding BARP; and d) a heterologous nucleic acid encoding CHAT.

In one embodiment, the invention relates to isolated recombinant cells comprising: a) a heterologous nucleic acid encoding an α6 subunit of an α6β4 nAChR; b) a heterologous nucleic acid encoding a β4 subunit of an α6β4 nAChR; c) a heterologous nucleic acid encoding BARP; and d) a heterologous nucleic acid encoding SULT2B1.

In one embodiment, the invention relates to isolated recombinant cells comprising: a) a heterologous nucleic acid encoding an α6 subunit of an α6β4 nAChR; b) a heterologous nucleic acid encoding a β4 subunit of an α6β4 nAChR; c) a heterologous nucleic acid encoding BARP; d) a heterologous nucleic acid encoding CHAT; and e) a heterologous nucleic acid encoding SULT2B1.

In one embodiment, the α6 subunit of the α6β4 nAChR comprises an amino acid sequence with at least 85%, or at least 90%, or at least 95%, such as 95%, 96%, 97%, 98%, 99%, or 100% identity to the amino acid sequence of SEQ ID NO: 1.

SEQ ID NO: 1
MLTSKGQGFLHGGLCLWLCVFTPFFKGCVGCATEERLFHKLFSHYNQFIR
PVENVSDPVTVHFEVAITQLANVDEVNQIMETNLWLRHIWNDYKLRWDPM
EYDGIETLRVPADKIWKPDIVLYNNAVGDFQVEGKTKALLKYNGMITWTP
PAIFKSSCPMDITFFPFDHQNCSLKFGSWTYDKAEIDLLIIGSKVDMNDF
WENSEWEIIDASGYKHDIKYNCCEEIYTDITYSFYIRRLPMFYTINLIIP
CLFISFLTVLVFYLPSDCGEKVTLCISVLLSLTVFLLVITETIPSTSLVV
PLVGEYLLFTMIFVTLSIVVTVFVLNIHYRTPTTHTMPRWVKTVFLKLLP
QVLLMRWPLDKTRGTGSDAVPRGLARRPAKGKLASHGEPRHLKECFHCHK
SNELATSKRRLSHQPLQWVVENSEHSPEVEDVINSVQFIAENMKSHNETK
EVEDDWKYVAMVVDRVFLWVFIIVCVFGTAGLFLQPLLGNTGKS

In one embodiment, the β4 subunit of the α6β4 nAChR comprises an amino acid sequence with at least 85%, or at least 90%, or at least 95%, such as 95%, 96%, 97%, 98%, 99%, or 100% identity to the amino acid sequence of SEQ ID NO: 2:

SEQ ID NO: 2
MRRAPSLVLFFLVALCGRGNCRVANAEEKLMDDLLNKTRYNNLIRPATSS
SQLISIKLQLSLAQLISVNEREQIMTTNVWLKQEWTDYRLTWNSSRYEGV
NILRIPAKRIWLPDIVLYNNADGTYEVSVYTNLIVRSNGSVLWLPPAIYK
SACKIEVKYFPFDQQNCTLKFRSWTYDHTEIDMVLMTPTASMDDFTPSGE
WDIVALPGRRTVNPQDPSYVDVTYDFIIKRKPLFYTINLIIPCVLTTLLA
ILVFYLPSDCGEKMTLCISVLLALTFFLLLISKIVPPTSLDVPLIGKYLM
FTMVLVTFSIVTSVCVLNVHHRSPSTHTMAPWVKRCFLHKLPTFLFMKRP
GPDSSPARAFPPSKSCVTKPEATATSTSPSNFYGNSMYFVNPASAASKSP
AGSTPVAIPRDFWLRSSGRFRQDVQEALEGVSFIAQHMKNDDEDQSVVED
WKYVAMVVDRLFLWVFMFVCVLGTVGLFLPPLFQTHAASEGPYAAQRD

In one embodiment, the BARP comprises an amino acid sequence with at least 85%, or at least 90%, or at least 95%, such as 95%, 96%, 97%, 98%, 99%, or 100% identity to the amino acid sequence of SEQ ID NO: 3. Or, the BARP comprises an amino acid sequence with at least 60%, at least 70%, at least 80%, at least 85%, or at least 90%, or at least 95%, such as 95%, 96%, 97%, 98%, 99%, or 100% identity to the amino acid sequence of SEQ ID NO: 3 and has protein chaperone property, which includes the property of enhancing the expression of the α6 and β4 subunits of α6β4 nAChR.

SEQ ID NO: 3
MQPTATMATAATTTTTTTATVALTTSWDNATGRPTAEPDPILDNYVLLVV
VMSLFVGGTLVVLSGVLLLCKRCWDVHQRLNRAMEEAEKTTTTYLDNGTH
PAQDPDFRGEDPECQDAETERFLSTSSTGRRVSFNEAALFEQSRKTQDKG
RRYTLTEGDFHHLKNARLTHLHLPPLKIVTIHECDSGEASSATTPHPATS
PKATLAIFQPPGKALTGRSVGPSSALPGDPYNSAAGATDFAEISPSASSD
SGEGTSLDAGTRSTKAGGPGAAAGPGEAGPGSGAGTVLQFLTRLRRHASL
DGASPYFKVKKWKLEPSQRAASLDTRGSPKRHHFQRQRAASESTEQEEGD
APQEDFIQYIARAGDAVAFPHPRPFLASPPPALGRLEAAEAAGGASPDSP
PERGAGSAGPEQQQPPLEPDAERDAGPEQAQTSYRDLWSLRASLELHAAA
SDHSSSGNDRDSVRSGDSSGSGSGGAAPAFPPPSPPAPRPKDGEARRLLQ
MDSGYASIEGRGAGDDTEPPAAPARPRSPRAWPRRPRRDYSIDEKTDALF
HEFLRHDPHFDDTPAAARHRARAHPHARKQWQRGRQHSDPGARAAPALAG
TPAPPAGAARPARAPLRRGDSVDGPPDGRTLGGAGDDPAIPVIEEEPGGG
GCPGSGLCVLPSGSVLDKLAAGLDERLFPPRLAEPVVATPALVAAAPTSP
DHSPA

In one embodiment, the CHAT comprises an amino acid sequence with at least 85%, or at least 90%, or at least 95%, such as 95%, 96%, 97%, 98%, 99%, or 100% identity to the amino acid sequence of SEQ ID NO: 4. Or, the CHAT comprises an amino acid sequence with at least 60%, at least 70%, at least 80%, at least 85%, or at least 90%, or at least 95%, such as 95%, 96%, 97%, 98%, 99%, or 100% identity to the amino acid sequence of SEQ ID NO: 4 and has protein chaperone property, which includes the property of enhancing the expression of the α6 and β4 subunits of α6β4 nAChR.

SEQ ID NO: 4
MAAKTPSSEESGLPKLPVPPLQQTLATYLQCMRHLVSEEQFRKSQAIVQQ
FGAPGGLGETLQQKLLERQEKTANWVSEYWLNDMYLNNRLALPVNSSPAV
IFARQHFPGTDDQLRFAASLISGVLSYKALLDSHSIPTDCAKGQLSGQPL
CMKQYYGLFSSYRLPGHTQDTLVAQNSSIMPEPEHVIVACCNQFFVLDVV
INFRRLSEGDLFTQLRKIVKMASNEDERLPPIGLLTSDGRSEWAEARTVL
VKDSTNRDSLDMIERCICLVCLDAPGGVELSDTHRALQLLHGGGYSKNGA
NRWYDKSLQFVVGRDGTCGVVCEHSPFDGIVLVQCTEHLLKHMTQSSRKL
IRADSVSELPAPRRLRWKCSPEIQGHLASSAEKLQRIVKNLDFIVYKFDN
YGKTFIKKQKCSPDAFIQVALQLAFYRLHRRLVPTYESASIRRFQEGRVD
NIRSATPEALAFVRAVTDHKAAVPASEKLLLLKDAIRAQTAYTVMAITGM
AIDNHLLALRELARAMCKELPEMFMDETYLMSNRFVLSTSQVPTTTEMFC
CYGPVVPNGYGACYNPQPETILFCISSFHSCKETSSSKFAKAVEESLIDM
RDLCSLLPPTESKPLATKEKATRPSQGHQP

In one embodiment, the SULT2B1 comprises an amino acid sequence with at least 85%, or at least 90%, or at least 95%, such as 95%, 96%, 97%, 98%, 99%, or 100% identity to the amino acid sequence of SEQ ID NO: 5. Or, the SULT2B1 comprises an amino acid sequence with at least 60%, at least 70%, at least 80%, at least 85%, or at least 90%, or at least 95%, such as 95%, 96%, 97%, 98%, 99%, or 100% identity to the amino acid sequence of SEQ ID NO: 5 and has protein chaperone property, which includes the property of enhancing the expression of the α6 and β4 subunits of α6β4 nAChR.

SEQ ID NO: 5
MDGPAEPQIPGLWDTYEDDISEISQKLPGEYFRYKGVPFPVGLYSLESIS
LAENTQDVRDDDIFIITYPKSGTTWMIEIICLILKEGDPSWIRSVPIWER
APWCETIVGAFSLPDQYSPRLMSSHLPIQIFTKAFFSSKAKVIYMGRNPR
DVVVSLYHYSKIAGQLKDPGTPDQFLRDFLKGEVQFGSWFDHIKGWLRMK
GKDNFLFITYEELQQDLQGSVERICGFLGRPLGKEALGSVVAHSTFSAMK
ANTMSNYTLLPPSLLDHRRGAFLRKGVCGDWKNHFTVAQSEAFDRAYRKQ
MRGMPTFPWDEDPEEDGSPDPEPSPEPEPKPSLEPNTSLEREPRPNSSPS
PSPGQASETPHPRPS

Any suitable means for introducing heterologous nucleic acid into a cell can be used herein to prepare the recombinant cells disclosed herein, such as DNA transfection (e.g., via a DNA vector) and RNA transduction. In one embodiment, the heterologous nucleic acid to be introduces into cells to generate the recombinant cells are prepared by using a vector, preferably an expression vector. The term “vector” refers to a nucleic acid molecule capable of transporting another nucleic acid to which it has been linked. One type of vector is a “plasmid”, which refers to a circular double-stranded DNA loop into which additional DNA segments can be inserted. Another type of vector is a viral vector wherein additional DNA segments can be inserted. Expression vectors are those vectors capable of directing the expression of genes to which they are operably linked. The expression vectors used herein comprise a nucleic acid encoding a protein sequence in a form suitable for expression of the nucleic acid in a host cell. Thus, the expression vectors can include one or more regulatory sequences, such as a promoter, selected on the basis of the host cells to be used for expression, operably linked to the nucleic acid sequence to be expressed. When used in reference to a expression vector, “operably linked” is intended to mean that the nucleotide sequence of interest is linked to the regulatory sequence(s) in a manner allowing for expression of the nucleotide sequence (e.g., in an in vitro transcription/translation system or in a host cell when the vector is introduced into the host cell). It will be appreciated by those of ordinary skill in the art that the design of the expression vector can depend on such factors as the choice of the host cell to be transformed and the level of expression of protein desired as well as the intended use of the vector.

Any vector known to those skilled in the art in view of the present disclosure can be used, such as a plasmid, a cosmid, a phage vector, or a viral vector. In one embodiment, the vector is an expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein. Conventional cloning techniques or artificial gene synthesis can be used to generate an expression vector according to embodiments of the invention.

Any cell known to those skilled in the art in view of the present disclosure can be used for recombinant expression of α6β4 nAChR, BARP, CHAT, and SULT2B1. In one embodiment, the recombinant cell is a mammalian cell. Suitable the mammalian cells may be selected from human embryonic kidney 293T (HEK293T) cell, HEK293F cells, Hela cells, Chinese hamster ovary (CHO) cells, NIH 3T3 cells, MCF-7 cells, Hep G2 cells, baby hamster kidney (BHK) cells, and Cos7 cells.

Methods of Identifying Agonists, Antagonists, or Positive Allosteric Modulators of α6β4 nAChR

Further provided herein are methods of identifying agonists, antagonists, or positive allosteric modulators (PAMs) of an α6β4 nAChR. PAMs are compounds that bind at sites on the protein surface other than the active sites, and therefore change the conformation of the protein binding sites.

In one embodiment, the method comprises culturing the isolated recombinant cells disclosed herein under conditions where the recombinant cells grow, contacting the recombinant cells with an agent, and determining if the agent is an agonist, antagonist, or PAM of the α6β4 nAChR, wherein an agonist or PAM enhances the activity of the α6β4 nAChR and an antagonist decreases the activity of the α6β4 nAChR as compared to the activity of the α6β4 nAChR in a recombinant cell that was not contacted with an agent. Agonists, as used herein, refer to molecules/compounds/peptides that serve to enhance the function of the α6β4 nAChR. PAMs, as used herein, refer to molecules/compounds/peptides that enhance the effect of α6β4 nAChR's response to a ligand without directly activating the receptor. As used herein, the term “enhance”, “enhanced”, “increase”, or “increased”, when used with respect to α6β4 nAChR activity refers to an increase in the signaling through the receptor, relative to the corresponding signaling observed in a cell in which an agonist or PAM is not administered. Antagonists, as used herein, refer to molecules/compounds/peptides that serve to block, decrease, or dampen the function of the α6β4 nAChR. In particular embodiments, the agent is a small molecule or peptide.

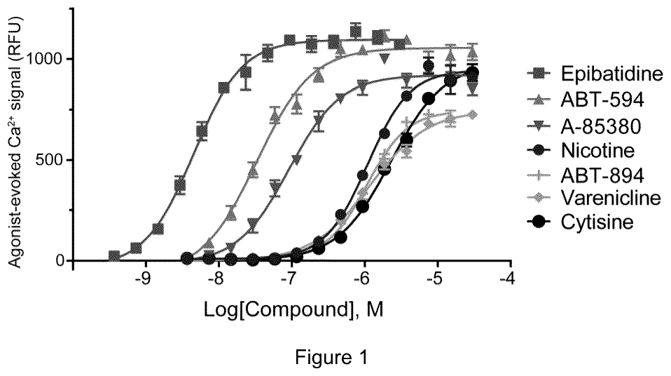

In one embodiment, FLIPR assay is used to identify agonists of α6β4 nAChR mediated calcium flux. In this assay, the recombinant cells are incubated with a calcium sensitive dye (such as Ca5), exposed to a test compound, and calcium flux is imaged by FLIPR^{TETRA}. As shown in FIG. 1, agonists of α6β4 nAChR enhances calcium flux.

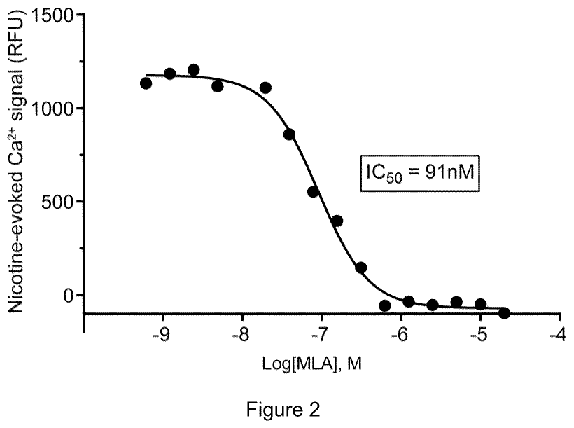

In one embodiment, FLIPR assay is used to identify antagonists of nicotine-evoked α6β4 nAChR mediated calcium flux. In this assay, the recombinant cells are incubated with a calcium sensitive dye (such as Ca5). Using a double addition protocol, the recombinant cells are exposed to test compounds during the first duration, and to nicotine (e.g., at EC₈₀ (15 μm)) during the second duration. Thereafter, the calcium flux is imaged by FLIPR^{TETRA}. As shown in FIG. 2, antagonists of α6β4 nAChR reduce calcium flux.

In one embodiment, FLIPR assay is used to identify compounds that positively modulate or potentiate nicotine-evoked α6β4 nAChR mediated calcium flux. In this assay, the recombinant cells are incubated with a calcium sensitive dye (such as Ca5). Using a double addition protocol, the recombinant cells are exposed to test compounds during the first duration, and to nicotine (e.g., at EC₈₀ (15 μm)) during the second duration. Thereafter, the calcium flux is imaged by FLIPR^{TETRA}. If a test compound enhances nicotine-evoked α6β4 nAChR mediated calcium flux, yet does not enhances calcium flux as an agonist (determined as above), the test compound is termed a positive allosteric modulator (PAM) or potentiator of α6β4 nAChR.

In a preferred embodiment, the cells are incubated at about 25-35° C. for about 20-50 hr prior to the FLIPR assay or other assay for measuring α6β4 nAChR activity. In some embodiments, the cells are incubated at about 25° C., about 26° C., about 27° C., about 28° C., about 29° C., about 30° C., about 31° C., about 32° C., about 33° C., about 34° C., or about 35° C., for about 20 hr, about 25 hr, about 30 hr, about 35 hr, about 40 hr, about 45 hr, or about 50 hr prior to the assay.

### Expression System and Kits

Further provided herein are expression systems and kits comprising the isolated recombinant cells. The expression systems and kits may further include instructions for use.

### EXAMPLE

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Material

| Name | Company | Cat.No |
|---|---|---|
| FreeStyle 293 Expression Media | Gibco | 12338018 |
| OptiPro SFM | Gibco | 12309019 |
| FreeStyle MAX Reagent | Invitrogen | 16447100 |
| Recovery Cell Culture Freezing Media | Gibco | 12648010 |
| DMEM-high glucose | Sigma | D5671-500 ml |
| Defined FBS | HyClone | SH30070.03 |
| Sodium Pyruvate | HyClone | SH30239.01 |
| Glutamax | HyClone | 35050061 |
| DMSO | Sigma Aldrich | 41648 |
| Ca5 dye | Molecular | R8186 |
| | Devices | |
| Nicotine | Tocris | 3546 |
| DHβe | Tocris | 2349 |
| 384-well pp plates | Greiner | 781280 |
| 384-well poly-D-lysine plates | Corning | 354663 |
| Custom HBSS | HyClone | SH3A4475.01 |
| Calcium Chloride | Amresco | E506-500 ML |
| Magnesium Chloride | Amresco | E525-500 ML |
| Freezing Vial | Nalgene | 5000-0020 |

### Cell Line

FreeStyle 293F cell line (ThermoFisher Scientific Cat #R79007)

### Vector Constructs

pcDNA3.1-α6
pcDNA3.1-84
pcDNA3.1-BARP
pcDNA3.1-SULT2B1
pcDNA3.1-CHAT

### Culturing Media

FreeStyle 293 Expression Medium
Transfection Reagents
FreeStyle MAX Reagent

### Planting Media

DMEM
10% FBS
1% pen-strep

### Compound Dilution/Ca5 Buffer.

HEPES-buffered saline solution (500 mL) supplemented with 1 mM Mg²⁺ and 2 mM Ca²⁺

### Calcium 5 (Ca5) Dye (Molecular Devices)

Calcium dye diluted at 25× concentration in Hanks Buffered Salt Solution. Dye diluted to 1× in HEPES assay buffer with 2 mM CaCl₂) and 1 mM MgCl₂.

### Methods and Procedures

### Day 1 Cell Preparation

Prepare a cell suspension of 1×10⁶ cells per mL by centrifuging cells and resuspending in fresh, prewarmed media;
With 1900 mL of cells shaking, prepare the following transfection mix in 50 ml of OptiPro SFM Medium;
Add 2.5 mL FreeStyle MAX reagent to 47.5 mL of OptiPro SFM to an empty 50 ml tube;
In a second 50 ml tube, add 2.5 mg total DNA (α6, β4, BARP, CHAT, and SULT2B1 at a ratio of 1:1:2:1:1) in OptiPro SFM to a final volume of 50 ml and vortex gently;
Transfer the diluted DNA to the FreeStyle MAX reagent and gently mix the solution immediately and incubate for 10 minutes at room temperature;
Transfer the 100 ml FreeStyle MAX/DNA transfection mix to the cells and homogenize the culture;
Incubate 1 hour at 37° C. with 8% CO₂;
Pour cells into sterile 500 mL tubes;
Spin at 1000 rpm for 7 minutes;
Remove supernatant; and
Resuspend Cells in Plating Media.

### Day 2 Plate Cells

Ethanol vials to decontaminate;
Pour cells into 50 ml conical tube;
Slowly add 10 ml warmed plating media drop wise;
Pipet up and down gently a few times to dislodge pellet if it exists;
Spin at 1000 RPM for 7 min, aspirate supernatant, and resuspend in 25 ml plating;
Seed 35,000 cells/well in 35 μL (1.0×10⁶ cells/mL); and
Incubate plates for 24 hours at 37° C. Then transfer the plate to 30° C. with 5% CO₂ in a humidified atmosphere for 24-48 hours.

### Day 3 FLIPR Assay

Wash plate with plate washer (4 washes×100 μL/wash) using compound dilution buffer, leaving 25 UL in each well;
Add 25 μL of 2× Ca6 dye to each well;
Incubate at room temperature for 1 hour;
Wash plate with plate washer (4 washes×100 μL/wash) using compound dilution buffer, leaving 50 UL in each well;
Transfer plates to FLIPR for compound addition.
   Concentration Response Curves for Agonist-Evoked Activity of α6β4 nAChR

Human α6 and β4 subunits of α6β4 nAChR were co-transfected with BARP, SULT2B1, and CHAT in HEK293T cells and incubated at 30° C. for 24-48 hours. The transfected cells were incubated for one hour at room temperature with Ca5 dye followed by stimulating with agonists for 3.5 minutes. Agonist-evoked calcium flux was measured using a FLIPR^{TETRA} imager and final FLIPR signals were averaged and plotted (FIG. 1). As shown, each of the agonists tested (epibatidine, ABT-594, A-85380, nicotine, ABT-894, varenicline, and cytisine) enhances calcium flux in a dose-dependent manner.

Concentration Response Curve for Antagonist on Nicotine-Evoked Activity of α6β4 nAChR

Human α6 and β4 subunits of α6β4 nAChR were co-transfected with BARP, SULT2B1, and CHAT in HEK293T cells and incubated at 30° C. for 24-48 hour. The transfected cells were incubated for one hour at room temperature with Ca5 dye followed by stimulation using a two-addition protocol (1^{st} addition: with methyllycaconitine (MLA) at various concentration for 3 min; 2^{nd} addition: with nicotine (EC₈₀, 15 μM) for 3.5 min). Nicotine-evoked calcium flux was then measured using a FLIPR^{TETRA} imager and the final FLIPR signals were averaged and plotted (FIG. 2). As shown, MLA (a known agonist of α6β4 nAChR) decreases the nicotine-evoked calcium flux in a dose-dependent manner.

Functional Expression of α6β4 nAChR

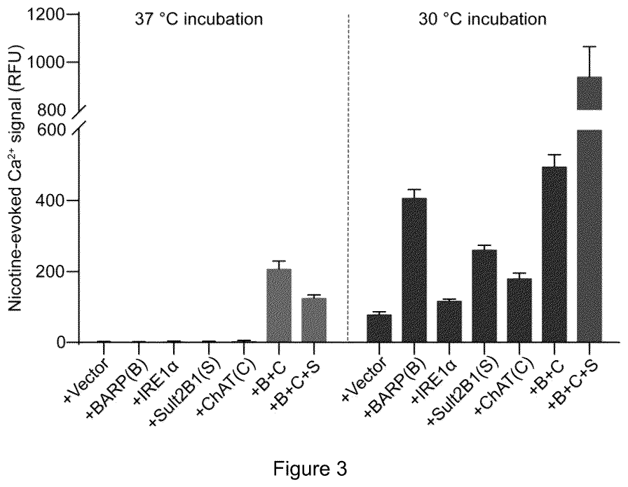

Human α6 and β4 subunits of α6β4 nAChR were co-transfected with specified combinations of cDNAs in HEK293T cells and incubated at either 30° C. or 37° C. for 24-48 hours. The transfected cells were incubated for one hour at room temperature with Ca5 dye followed by stimulation with Eₘₐₓ nicotine (66 M). Nicotine-evoked Ca²⁺ signal of the transfected are graphed in FIG. 3. As shown, with incubation at 37° C., co-transfection with BARP and CHAT or with BARP, CHAT, and SULT2B1 enhances functional expression of α6β4 nAChR. With incubation at 30° C., co-transfection with BARP, CHAT, or SULT2B1, both individually or in combination, enhances functional expression of α6β4 nAChR.

What is claimed is:

1. An isolated recombinant cell comprising:
a) a heterologous nucleic acid encoding an α6 subunit of an α6β4 nicotinic acetylcholine receptor (nAChR);
b) a heterologous nucleic acid encoding a β4 subunit of an α6β4 nAChR; and
c) at least one heterologous nucleic acid selected from the group consisting of a heterologous nucleic acid encoding beta associated regulatory protein (BARP), a heterologous nucleic acid encoding choline O-acetyltransferase (CHAT), and a heterologous nucleic acid encoding sulfotransferase family 2B member 1 (SULT2B1).

2. The isolated recombinant cell of claim 1, wherein the recombinant cell comprises the heterologous nucleic acid encoding BARP, the heterologous nucleic acid sequence encoding CHAT, and the heterologous nucleic acid sequence encoding SULT2B1.

3. The isolated recombinant cell of claim 1, wherein the recombinant cell is a mammalian cell.

4. The isolated recombinant cell of claim 3, wherein the mammalian cell is selected from the group consisting of a human embryonic kidney 293T (HEK293T) cell, a HEK293F cell, a HeLa cell, a Chinese hamster ovary (CHO) cell, a NIH 3T3 cell, a MCF-7 cell, a Hep G2 cell, a baby hamster kidney (BHK) cell, and a Cos7 cell.

5. The isolated recombinant cell of claim 1, wherein the α6 subunit of the α6β4 nAChR comprises an amino acid sequence with at least 95% identity to the amino acid sequence of SEQ ID NO: 1.

6. The isolated recombinant cell of claim 1, wherein the β4 subunit of the α6β4 nAChR comprises an amino acid sequence with at least 95% identity to the amino acid sequence of SEQ ID NO: 2.

7. The isolated recombinant cell of claim 1, wherein the BARP comprises an amino acid sequence with at least 95% identity to the amino acid sequence of SEQ ID NO: 3.

8. The isolated recombinant cell of claim 1, wherein the CHAT comprises an amino acid sequence with at least 95% identity to the amino acid sequence of SEQ ID NO: 4.

9. The isolated recombinant cell of claim 1, wherein the SULT2B1 comprises an amino acid sequence with at least 95% identity to the amino acid sequence of SEQ ID NO: 5.

10. A method for identifying agonists, antagonists, or positive allosteric modulators of α6β4 nAChR, the method comprising:
a) contacting the isolated recombinant cell of claim 1 with an agent; and
b) determining the activity of the α6β4 nAChR of the isolated recombinant cell, wherein the agent is identified as an agonist or positive allosteric modulator (PAM) if the agent enhances the activity of the α6β4 nAChR and the agent is identified as an antagonist if the agent decreases the activity of the α6β4 nAChR as compared to the activity of the α6β4 nAChR when the isolated recombinant cell was not contacted with the agent.

11. The method of claim 10, wherein step b) comprises determining calcium flux of the isolated recombinant cell, wherein the agent is identified as an agonist if the agent enhances the calcium flux as compared to the calcium flux when the isolated recombinant cell was not contacted with the agent.

12. The method of claim 10, wherein step b) comprises determining calcium flux and nicotine-evoked calcium flux of the isolated recombinant cell, wherein the agent is identified as an PAM if the agent does not enhance calcium flux and enhances the nicotine-evoked calcium flux as compared to the calcium flux and nicotine-evoked calcium flux when the isolated recombinant cell was not contacted with the agent

13. The method of claim 10, wherein step b) comprises determining nicotine-evoked calcium flux of the isolated recombinant cell, wherein the agent is identified as an antagonist if the agent decreases the nicotine-evoked calcium flux as compared to the nicotine-evoked calcium flux when the isolated recombinant cell was not contacted with the agent.

14. The method of claim 10, wherein the isolated recombinant cell is incubated at about 25° C.-35° C. for about 20-50 hours prior to being contacted with the agent.

15. The method of claim 10, wherein the agent is a small molecule or peptide.

16. A kit comprising (i) the isolated recombinant cell of claim 1, and (ii) instructions for use.


SEQUENCE LISTING (5 sequences)

SEQ ID NO: 1 - protein, 494 aa, Homo sapiens
MLTSKGQGFLHGGLCLWLCVFTPFFKGCVGCATEERLFHKLFSHYNQFIRPVENVSDPVTVHFEVAITQLANVDEVNQIMETNLWLRHIWNDYKLRWDPMEYDGIETLRVPADKIWKPDIVLYNNAVGDFQVEGKTKALLKYNGMITWTPPAIFKSSCPMDITFFPFDHQNCSLKFGSWTYDKAEIDLLIIGSKVDMNDFWENSEWEIIDASGYKHDIKYNCCEEIYTDITYSFYIRRLPMFYTINLIIPCLFISFLTVLVFYLPSDCGEKVTLCISVLLSLTVFLLVITETIPSTSLVVPLVGEYLLFTMIFVTLSIVVTVFVLNIHYRTPTTHTMPRWVKTVFLKLLPQVLLMRWPLDKTRGTGSDAVPRGLARRPAKGKLASHGEPRHLKECFHCHKSNELATSKRRLSHQPLQWVVENSEHSPEVEDVINSVQFIAENMKSHNETKEVEDDWKYVAMVVDRVFLWVFIIVCVFGTAGLFLQPLLGNTGKS

SEQ ID NO: 2 - protein, 498 aa, Homo sapiens
MRRAPSLVLFFLVALCGRGNCRVANAEEKLMDDLLNKTRYNNLIRPATSSSQLISIKLQLSLAQLISVNEREQIMTTNVWLKQEWTDYRLTWNSSRYEGVNILRIPAKRIWLPDIVLYNNADGTYEVSVYTNLIVRSNGSVLWLPPAIYKSACKIEVKYFPFDQQNCTLKFRSWTYDHTEIDMVLMTPTASMDDFTPSGEWDIVALPGRRTVNPQDPSYVDVTYDFIIKRKPLFYTINLIIPCVLTTLLAILVFYLPSDCGEKMTLCISVLLALTFFLLLISKIVPPTSLDVPLIGKYLMFTMVLVTFSIVTSVCVLNVHHRSPSTHTMAPWVKRCFLHKLPTFLFMKRPGPDSSPARAFPPSKSCVTKPEATATSTSPSNFYGNSMYFVNPASAASKSPAGSTPVAIPRDFWLRSSGRFRQDVQEALEGVSFIAQHMKNDDEDQSVVEDWKYVAMVVDRLFLWVFMFVCVLGTVGLFLPPLFQTHAASEGPYAAQRD

SEQ ID NO: 3 - protein, 705 aa, Homo sapiens
MQPTATMATAATTTTTTTATVALTTSWDNATGRPTAEPDPILDNYVLLVVVMSLFVGGTLVVLSGVLLLCKRCWDVHQRLNRAMEEAEKTTTTYLDNGTHPAQDPDFRGEDPECQDAETERFLSTSSTGRRVSFNEAALFEQSRKTQDKGRRYTLTEGDFHHLKNARLTHLHLPPLKIVTIHECDSGEASSATTPHPATSPKATLAIFQPPGKALTGRSVGPSSALPGDPYNSAAGATDFAEISPSASSDSGEGTSLDAGTRSTKAGGPGAAAGPGEAGPGSGAGTVLQFLTRLRRHASLDGASPYFKVKKWKLEPSQRAASLDTRGSPKRHHFQRQRAASESTEQEEGDAPQEDFIQYIARAGDAVAFPHPRPFLASPPPALGRLEAAEAAGGASPDSPPERGAGSAGPEQQQPPLEPDAERDAGPEQAQTSYRDLWSLRASLELHAAASDHSSSGNDRDSVRSGDSSGSGSGGAAPAFPPPSPPAPRPKDGEARRLLQMDSGYASIEGRGAGDDTEPPAAPARPRSPRAWPRRPRRDYSIDEKTDALFHEFLRHDPHFDDTPAAARHRARAHPHARKQWQRGRQHSDPGARAAPALAGTPAPPAGAARPARAPLRRGDSVDGPPDGRTLGGAGDDPAIPVIEEEPGGGGCPGSGLCVLPSGSVLDKLAAGLDERLFPPRLAEPVVATPALVAAAPTSPDHSPA

SEQ ID NO: 4 - protein, 630 aa, Homo sapiens
MAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHMTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP

SEQ ID NO: 5 - protein, 365 aa, Homo sapiens
MDGPAEPQIPGLWDTYEDDISEISQKLPGEYFRYKGVPFPVGLYSLESISLAENTQDVRDDDIFIITYPKSGTTWMIEIICLILKEGDPSWIRSVPIWERAPWCETIVGAFSLPDQYSPRLMSSHLPIQIFTKAFFSSKAKVIYMGRNPRDVVVSLYHYSKIAGQLKDPGTPDQFLRDFLKGEVQFGSWFDHIKGWLRMKGKDNFLFITYEELQQDLQGSVERICGFLGRPLGKEALGSVVAHSTFSAMKANTMSNYTLLPPSLLDHRRGAFLRKGVCGDWKNHFTVAQSEAFDRAYRKQMRGMPTFPWDEDPEEDGSPDPEPSPEPEPKPSLEPNTSLEREPRPNSSPSPSPGQASETPHPRPS